(19) United States
(12) Patent Application Publication
(10) Pub. No.: US 2026/0215683 A1
(43) Pub. Date: Jul. 30, 2026

(54) WIDE-ANGLE VIEWING SYSTEM FOR AN OPHTHALMIC MICROSCOPE

(71) Applicant: Alcon Inc., Fribourg (CH)

(72) Inventors: Christopher Paul VOITA, Dove Canyon, CA (US); Federico PENNACCHINI, Irvine, CA (US); Sumit PALIWAL, Irvine, CA (US); Richard J. MICHAELS, Irvine, CA (US); Douglas ALLEN, Santa Barbara, CA (US); Rafael CHAN, Arlington, TX (US)

(21) Appl. No.: 19/398,532

(22) Filed: Nov. 24, 2025

Related U.S. Application Data
(60) Provisional application No. 63/750,118, filed on Jan. 27, 2025.

(51) Int. Cl. A61B 3/13; G02B 21/00

(52) U.S. Cl. CPC A61B 3/13; G02B 21/0012

(57) ABSTRACT

A wide-angle viewing system (WAVS) for an ophthalmic microscope is provided. The WAVS includes a frame, a reduction lens module attached to the frame, and an image inverter-reverter module movably coupled to the frame. The reduction lens module includes an ophthalmic microscope mount, and a movable reduction lens assembly with an optical axis, a disengaged position, and an engaged position. The image inverter-reverter module includes an optical prism and a loupe lens assembly. The image inverter-reverter module has an optical axis, a stowed position, and a deployed position. When the movable reduction lens assembly is disposed in the engaged position, the optical axis of the movable reduction lens assembly is aligned with the optical axis of the ophthalmic microscope. When the image inverter-reverter module is disposed in the deployed position, the optical axis of the image inverter-reverter module is aligned with the optical axis of the ophthalmic microscope.

### BACKGROUND

The present disclosure relates to ophthalmic surgery. More particularly, the present disclosure relates to a wide-angle viewing system (WAVS) for an ophthalmic microscope.

Ophthalmic microscopes are essential to many ophthalmic surgeries, allowing the surgeon to perform the procedure safely, precisely, and efficiently. Ophthalmic microscopes provide high contrast and detailed imaging of the different regions of the human eye, and may include or support a variety of features, such as advanced visualization, customizable illumination, high-quality imaging at lower illumination levels, instrument connectivity, etc. Adding a WAVS to the ophthalmic microscope generally provides a panoramic or wide-angle view of the surgical field to the surgeon, such as the fundus of the eye.

### SUMMARY

Certain embodiments of the present disclosure advantageously provide a WAVS for an ophthalmic microscope.

In certain embodiments, the WAVS (apparatus) includes a frame, a reduction lens module attached to the frame, and an image inverter-reverter module movably coupled to the frame. The reduction lens module includes a mount configured to be attached to an ophthalmic microscope, and a movable reduction lens assembly that includes an adjustable reduction lens. The movable reduction lens assembly has an optical axis, a disengaged position, and an engaged position. The image inverter-reverter module includes an optical prism and a loupe lens assembly. The image inverter-reverter module has an optical axis, a stowed position, and a deployed position. When the mount is attached to the ophthalmic microscope and the movable reduction lens assembly is disposed in the engaged position, the optical axis of the movable reduction lens assembly is aligned with an optical axis of the ophthalmic microscope. When the mount is attached to the ophthalmic microscope and the image inverter-reverter module is disposed in the deployed position, the optical axis of the image inverter-reverter module is aligned with the optical axis of the ophthalmic microscope.

### BRIEF DESCRIPTION OF THE DRAWINGS

FIGS. 1A, 1B, and 1C depict an example WAVS, in accordance with embodiments of the present disclosure.

FIGS. 2A, 2B, and 2C depict a portion of a frame of the example WAVS, in accordance with embodiments of the present disclosure.

FIGS. 3A to 3H depict a portion of a frame of the example WAVS, in accordance with embodiments of the present disclosure.

FIGS. 4A and 4B depict an upper portion of the example WAVS, in accordance with embodiments of the present disclosure.

FIG. 4C depicts a sectional view of a reduction lens module of the example WAVS, in accordance with embodiments of the present disclosure.

FIGS. 4D, 4E, and 4F depict views of the reduction lens module of the example WAVS, in accordance with embodiments of the present disclosure.

FIG. 4G depicts the reduction lens module of the example WAVS, in accordance with embodiments of the present disclosure.

FIGS. 4H depicts a sectional view of the reduction lens module of the example WAVS, in accordance with embodiments of the present disclosure.

FIG. 4I depicts a portion of FIG. 4H, in accordance with embodiments of the present disclosure.

FIGS. 5A to 5E depict a reduction lens assembly of the example WAVS, in accordance with embodiments of the present disclosure.

FIGS. 6A, 6B, and 6C depict a lower portion of the example WAVS, in accordance with embodiments of the present disclosure.

FIG. 6D depicts an articulated support arm of the example WAVS, in accordance with embodiments of the present disclosure.

FIG. 6E depicts the articulated support arm of FIG. 6D, in accordance with embodiments of the present disclosure.

FIG. 6F depicts a portion of FIG. 6D, in accordance with embodiments of the present disclosure.

FIG. 6G depicts a portion of FIG. 6E, in accordance with embodiments of the present disclosure.

FIG. 7 depicts a lower portion of the example WAVS, in accordance with embodiments of the present disclosure.

FIG. 8A depicts a lower portion of an image inverter-reverter module of the example WAVS, in accordance with embodiments of the present disclosure.

FIG. 8B depicts the lower portion of the image inverter-reverter module of FIG. 8A, in accordance with embodiments of the present disclosure.

FIG. 8C depicts a loupe lens assembly of the example WAVS, in accordance with embodiments of the present disclosure.

FIG. 8D depicts a lower portion of the image inverter-reverter module of FIG. 8A, in accordance with embodiments of the present disclosure.

FIG. 8E depicts a loupe lens assembly of the example WAVS, in accordance with embodiments of the present disclosure.

FIG. 8F depicts a loupe lens assembly of the example WAVS, in accordance with embodiments of the present disclosure.

FIG. 8G depicts a sectional view of the loupe lens assembly of FIG. 8F, in accordance with embodiments of the present disclosure.

FIGS. 8H, 8I, and 8J depict the loupe lens assembly of FIG. 8F in various positions, in accordance with embodiments of the present disclosure.

FIG. 8K depicts a portion of FIG. 8I, in accordance with embodiments of the present invention.

FIG. 9A depicts a ray trace diagram for an example ophthalmic microscope, in accordance with embodiments of the present disclosure.

FIG. 9B depicts a ray trace diagram for an example ophthalmic microscope with the example WAVS, in accordance with embodiments of the present disclosure.

FIG. 9C depicts an example optical prism for the example image inverter-reverter module, in accordance with embodiments of the present disclosure.

### DETAILED DESCRIPTION

Generally, a WAVS for an ophthalmic microscope includes a lens system and a separate inverter. The lens system can provide a wide-angle view of a fundus of an eye, while the inverter inverts the image of the fundus, including the retina, macula, optic disc, fovea and blood vessels. The inverter may be needed for certain procedures, such as indirect vitrectomy, but may not be needed for other surgical procedures, such as direct vitrectomy.

The lens system may be a contact lens type or a non-contact lens type. The lens system for the contact lens type includes a contact lens that is placed on a patient’s cornea. The lens system for the non-contact lens type includes one or more lenses that are positioned between the ophthalmic microscope and the patient’s cornea. For example, the lens system may include a frame that supports a reduction lens located proximate to the ophthalmic microscope, and a movable loupe lens located proximate to the patient’s cornea. The loupe lens may be moved along the optical axis of the ophthalmic microscope by a user, such as a surgeon, a surgical assistant, etc.

The inverter is mounted in an optical stack of the ophthalmic microscope between the ocular lenses (or eyepieces) and a lower portion of the microscope body, which may include a zoom module, an illumination module, etc. Unfortunately, mounting the inverter in the optical stack of the ophthalmic microscope raises the stack height, which commensurately raises the height of the ocular lenses of the ophthalmic microscope above the patient. Raising the eyepieces degrades the ergonomics of the ophthalmic microscope, particularly for users of shorter stature.

Certain embodiments of the present disclosure advantageously provide a WAVS for an ophthalmic microscope that integrates a lens system and an inverter into a single, configurable apparatus that enhances posterior segment visualization for certain ophthalmic procedures, such as posterior vitreoretinal procedures, etc., while maintaining the original ergonomics and performance of the ophthalmic microscope, such as stack height, focal length and working distance of the ophthalmic microscope. The integrated WAVS apparatus provides a more robust and precise opto-mechanical deployment and alignment during the surgical procedure.

Advantageously, the WAVS may be configured to operate in non-contact lens mode and contact lens mode for both indirect vitrectomy and direct vitrectomy. The WAVS also accommodates additional mechanical mechanisms (such as location holding and positioning mechanisms, etc.) for other procedures, such as minimally invasive glaucoma surgery (MIGS), etc.

In certain embodiments, the WAVS includes a frame, a reduction lens module attached to the frame, and an image inverter-reverter module movably coupled to the frame. The reduction lens module may include a movable reduction lens assembly with an adjustable reduction lens. Additionally or alternatively, the image inverter-reverter module may include an optical prism and a removable loupe lens assembly with a loupe lens.

The reduction lens assembly is movable between disengaged and engaged positions within the reduction lens module. In the disengaged position, the adjustable reduction lens is located outside of an optical axis of the ophthalmic microscope, while in the engaged position, the adjustable reduction lens is located within the optical axis.

The image inverter-reverter module is movable between a stowed position and a deployed position. In the stowed position, the optical prism and the loupe lens are located outside of the optical axis of the ophthalmic microscope. In the deployed position, the optical prism and the loupe lens are located within the optical axis. The loupe lens assembly may be detached from the image inverter-reverter module for sterilization or replacement purposes.

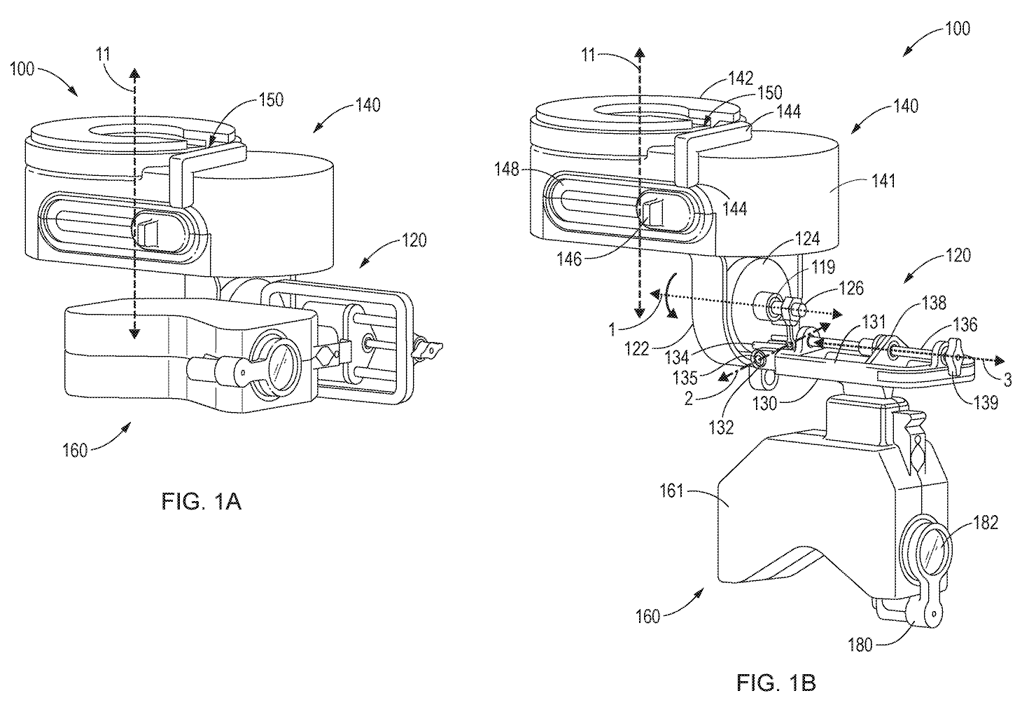

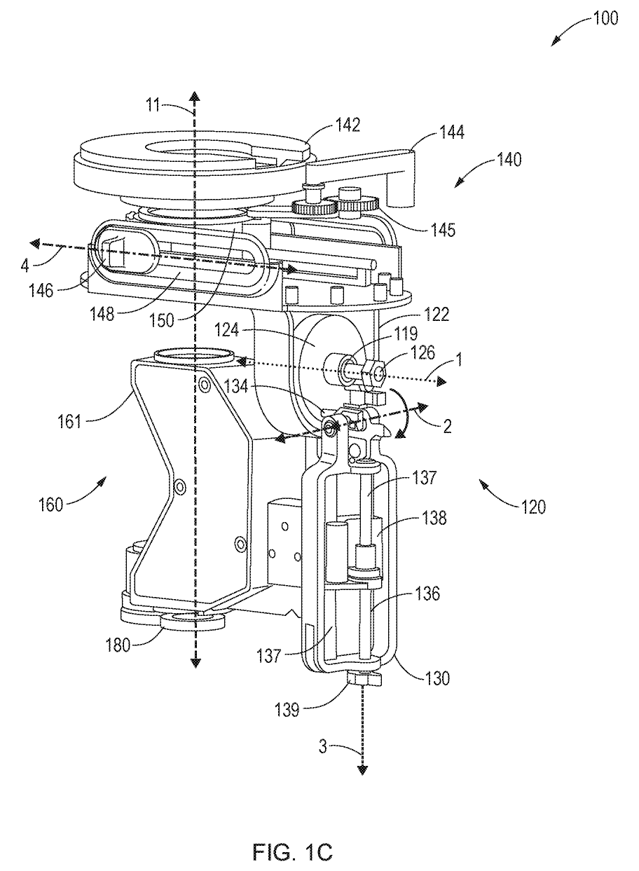

FIGS. 1A, 1B, and 1C depict WAVS 100, in accordance with embodiments of the present disclosure.

In certain embodiments, WAVS 100 includes frame 120, reduction lens module 140, reduction lens assembly 150, and image inverter-reverter module 160. Generally, reduction lens module 140 may be removably mounted to an ophthalmic microscope. Frame 120 is attached to and supports reduction lens module 140 and image inverter-reverter module 160, and allows image inverter-reverter module 160 to be stowed out of an optical path of the ophthalmic microscope, and then rotated into alignment with the optical path of the ophthalmic microscope.

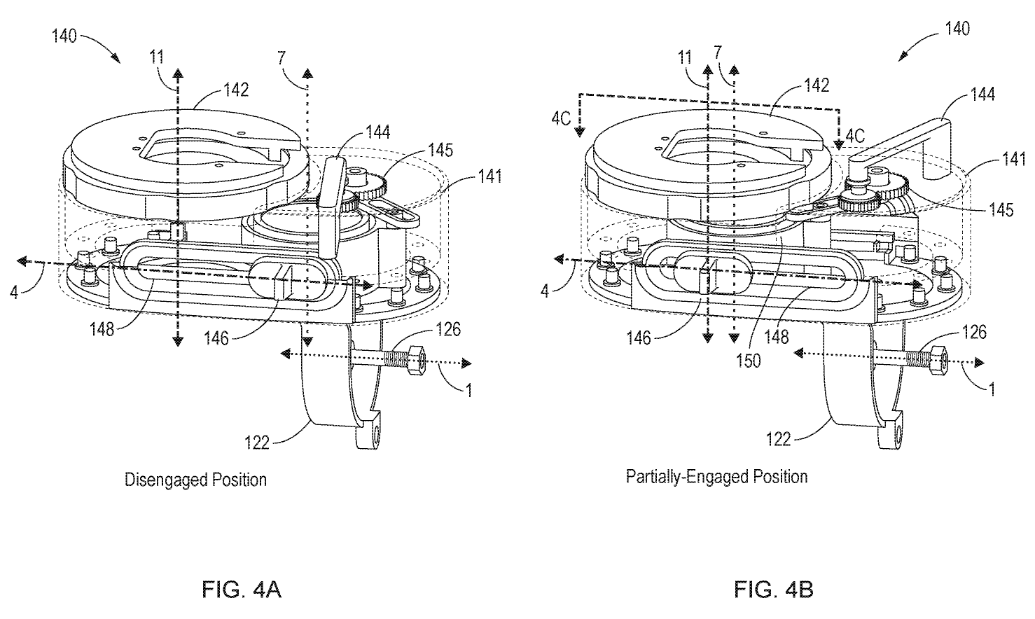

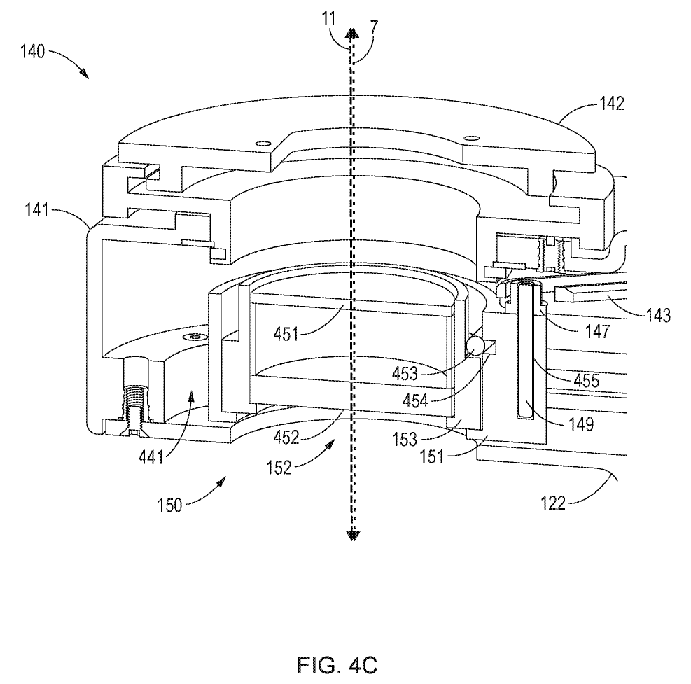

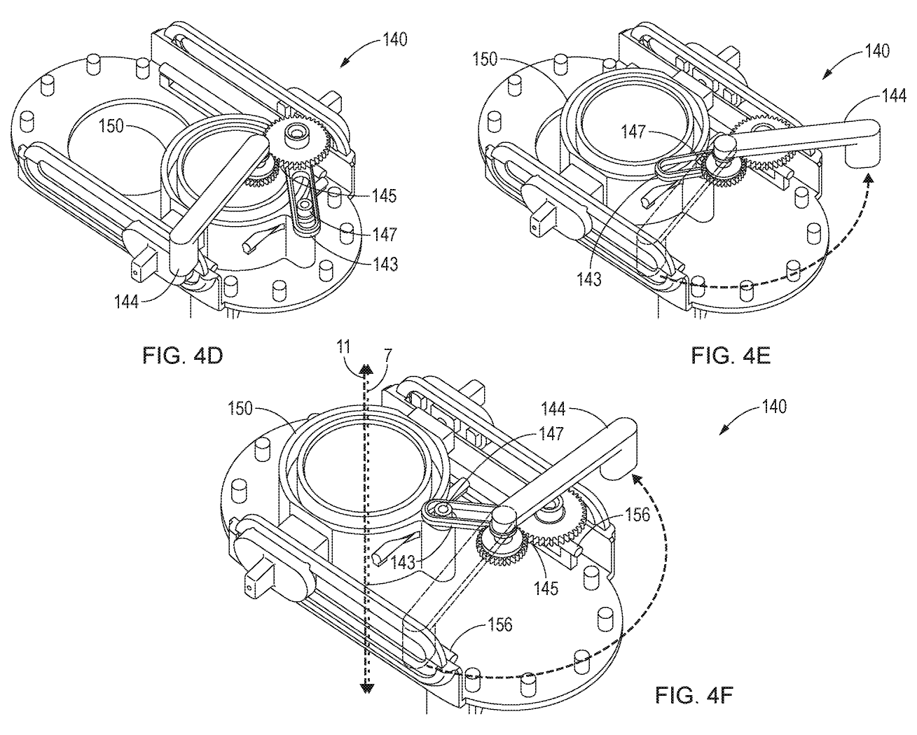

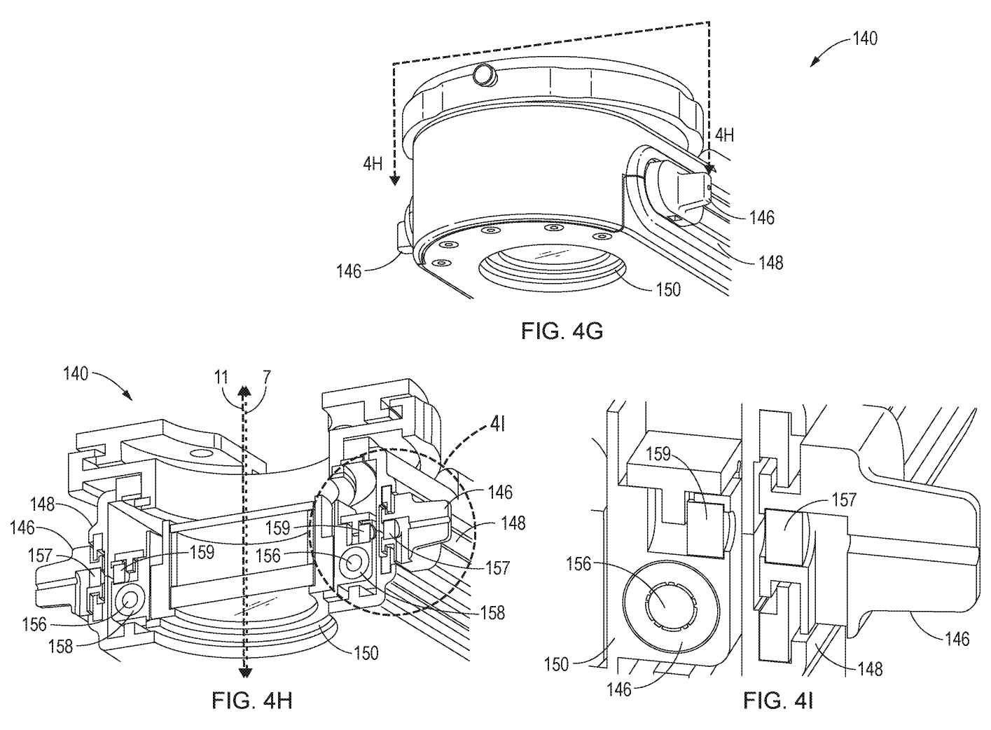

More particularly, reduction lens module 140 includes reduction lens assembly 150 (FIG. 1C) that is moveable between a disengaged position (FIGS. 1A, 1B, 4D) and an engaged position (FIGS. 1C, 4F). In the disengaged position, reduction lens module 140 is not located within the optical path of the ophthalmic microscope, while in the engaged position, reduction lens module 140 is located within the optical path of the ophthalmic microscope. Similarly, image inverter-reverter module 160 is movable between a stowed position (FIG. 1A), a partial stowed position (FIG. 1B), and a deployed position (FIG. 1C). Image inverter-reverter module 160 may be retained in the stowed, partial stowed, and deployed positions using any of cooperating magnets, mechanical locking mechanisms, interference fits, etc., and/or combinations thereof. In the stowed or partial stowed positions, image inverter-reverter module 160 is not located within the optical path of the ophthalmic microscope, while in the deployed position, image inverter-reverter module 160 is located within the optical path of the ophthalmic microscope. When reduction lens module 140 is disposed in the engaged position and image inverter-reverter module 160 is disposed in the deployed position, WAVS 100 provides a wide field of view for the ophthalmic microscope.

Frame 120 includes base 122 and articulated support arm 130. Base 122 includes rotation plate 124 coupled to shaft 126 via bearing 119. Shaft 126 extends from rotation plate 124, and defines a 1^{st} axis 1 for rotation of articulated support arm 130 and image inverter-reverter module 160. The 1^{st} axis 1 is perpendicular to an optical axis 11 of the ophthalmic microscope when WAVS 100 is installed. Base 122 may be attached to reduction lens module 140 using fasteners (such as bolts, screws, etc.), soldering, welding, etc. Articulated support arm 130 may be coupled to base 122 such that it can be moved and/or rotated relative to base 122. Articulated support arm 130 includes body 131, hinge 132, bushing assembly 134 coupled to hinge 132, threaded shaft 136, movable cradle 138, and knob 139.

Hinge 132 defines a 2^{nd} axis 2 for rotation of articulated support arm 130 and image inverter-reverter module 160, while threaded shaft 136 defines a 3^{rd} axis 3 for translation of cradle 138 and image inverter-reverter module 160. The 2^{nd} axis 2 extends through pin 135 of the hinge 132 and the 3^{rd} axis 3 extends along a length of the threaded shaft 136. The 2^{nd} axis 2 is perpendicular to the 1^{st} axis 1, and the 3^{rd} axis 3 is perpendicular to the 2^{nd} axis 2. In the partial stowed position (e.g., as illustrated in FIG. 1B), the 3^{rd} axis 3 is parallel to the 1^{st} axis 1.

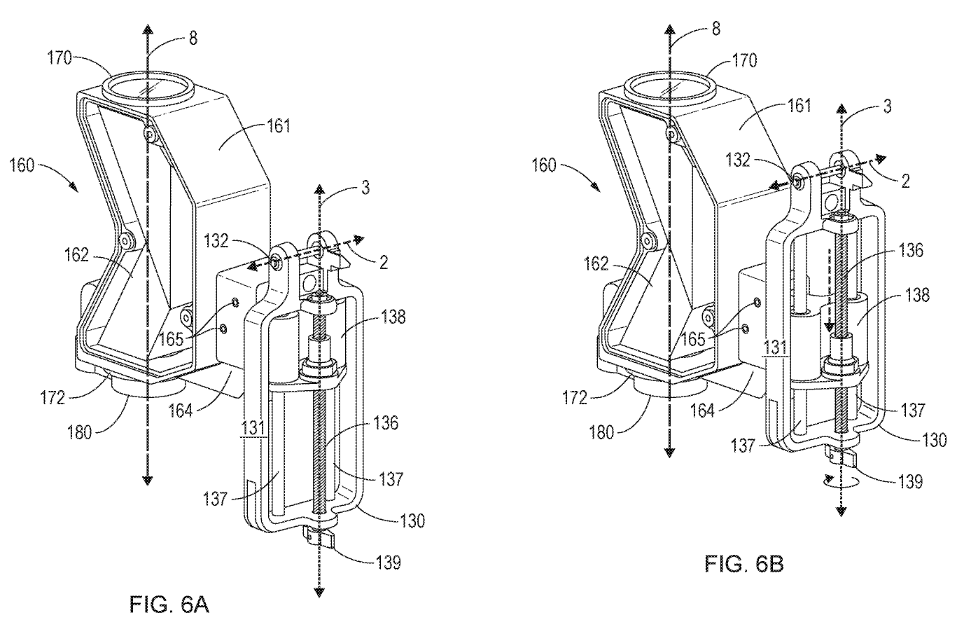

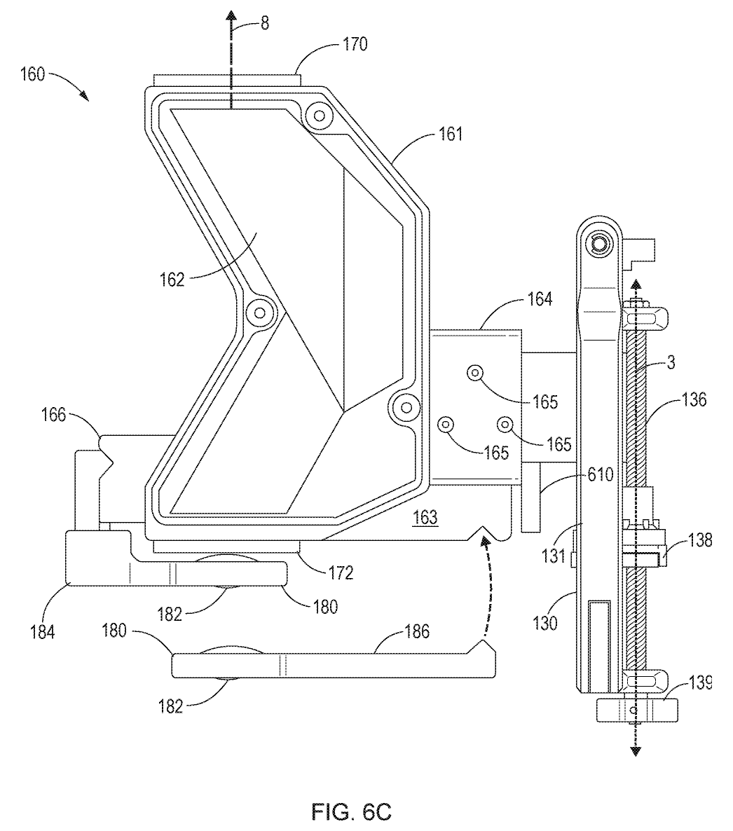

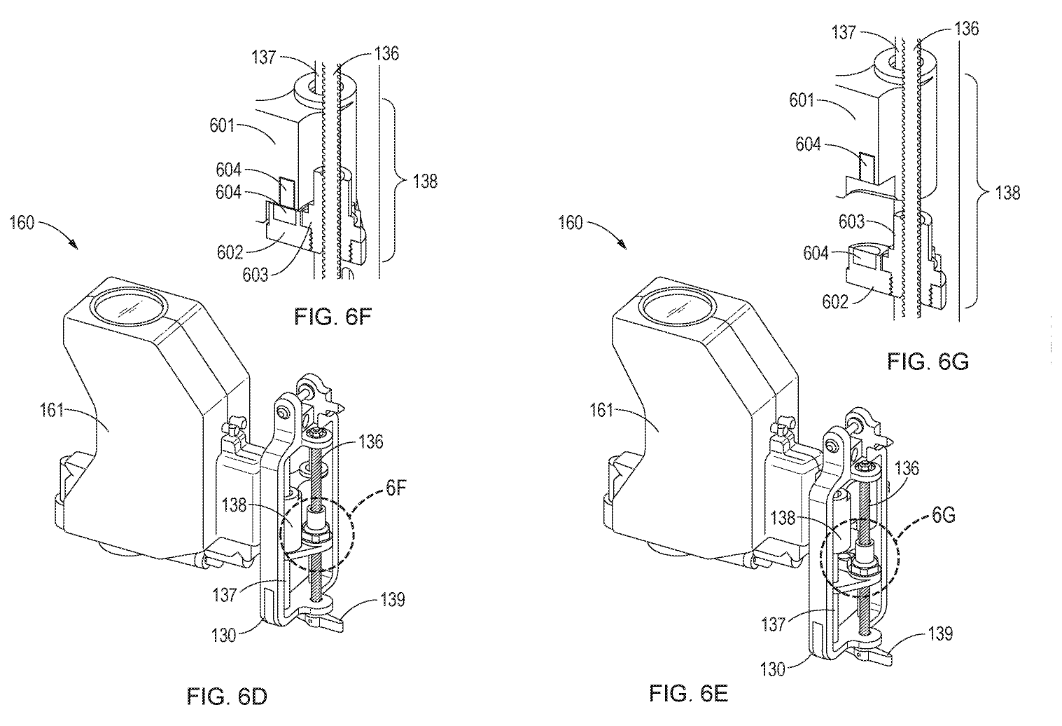

Cradle 138 is attached to image inverter-reverter module 160, and includes a threaded portion 603 (FIGS. 6D, 6E) through which threaded shaft 136 passes. Knob 139 rotates threaded shaft 136, which translates cradle 138 (and image inverter-reverter module 160) along the 3^{rd} axis 3 over a length of body 131. In certain embodiments, articulated support arm 130 may include one or more guide rods 137 (FIGS. 6A, 6B), disposed parallel to threaded shaft 136, to support cradle 138.

Generally, articulated support arm 130 and image inverter-reverter module 160 may be rotated about the 1^{st} axis 1 from the stowed position (FIG. 1A) to the partial stowed position (FIG. 1B), and vice versa.

Reduction lens module 140 includes reduction lens assembly 150 with one or more reduction lenses that cooperate with a loupe lens attached to the image inverter-reverter module 160 to adjust a focal plane of the ophthalmic microscope from the cornea to the retina and to increase the field of view, as discussed in more detail below. Reduction lens module 140 includes housing 141, mount 142, slider 146, channel 148, and reduction lens assembly 150. Slider 146 is attached to reduction lens assembly 150 using a fastener, such as a screw, a bolt, a pin, a pair of cooperating magnets (FIG. 4H), etc. In certain embodiments, an additional slider 146 and channel 148 are located on the other side of reduction lens module 140 (FIG. 4H).

Mount 142 is configured to be attached to a lower portion of the ophthalmic microscope, and may include a threaded mount, a bayonet mount, etc. In certain embodiments, mount 142 may include a bayonet mount that is configured to be attached to an illumination module of the ophthalmic microscope. In certain embodiments, reduction lens module 140 may also include lever arm 144 and gear train 145 (FIG. 1C), to facilitate translation of reduction lens assembly 150, as discussed in further detail below.

Reduction lens assembly 150 is moveable between a disengaged position (FIGS. 1A, 1B, 4D) and an engaged position (FIG. 1C, 4F). In the disengaged position, reduction lens assembly 150 is not located within the optical path of the ophthalmic microscope, allowing the ophthalmic microscope to be used conventionally, to be focused on the cornea, etc. Conversely, in the engaged position, reduction lens assembly 150 is located within the optical path of the ophthalmic microscope for use in combination with image inverter-reverter module 160. In other words, the location of reduction lens assembly 150 and image inverter-reverter module 160 within the optical path of the ophthalmic microscope provide a wide-angle field of view of the retina with the correct focus and image presentation.

More particularly, because the image of the retina is inverted and reverted when viewed through the cornea/lens of the eye, image inverter-reverter module 160 contains an optical prism, as discussed below, that compensates for these effects by inverting and reverting the image of retina viewed by the ophthalmic microscope.

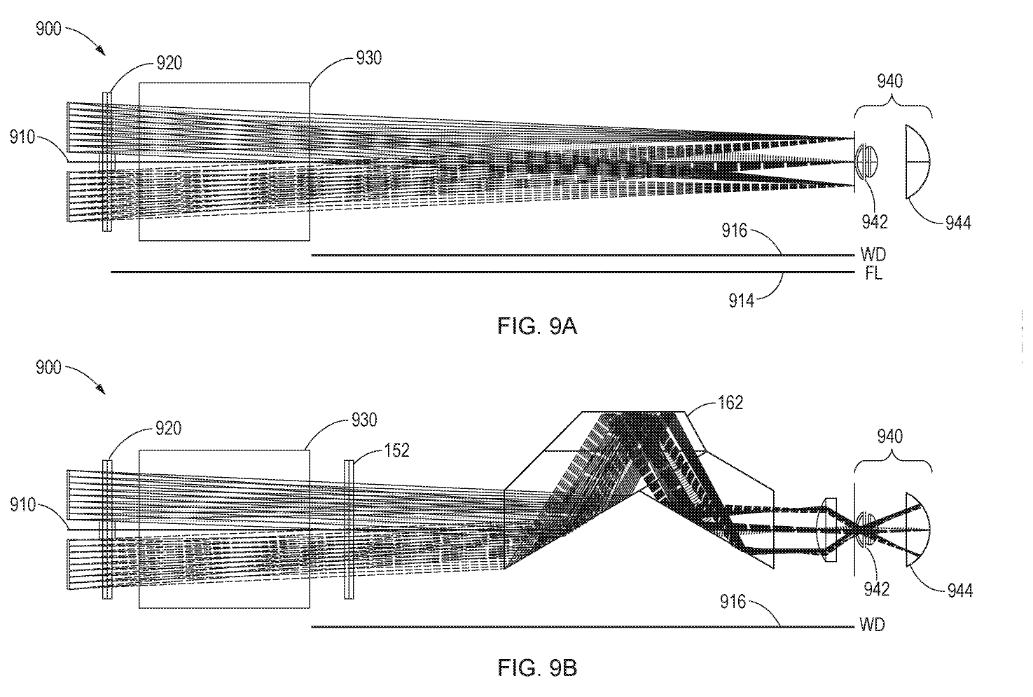

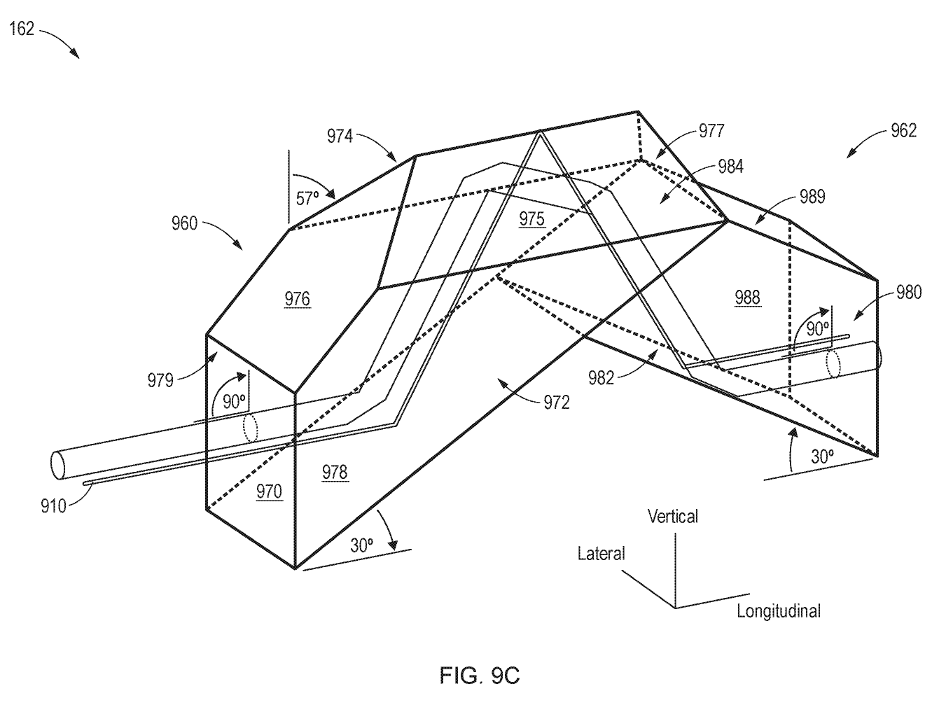

Image inverter-reverter module 160 may be coupled to articulated support arm 130 such that it can be moved and/or rotated relative to base 122. Image inverter-reverter module 160 includes housing 161, optical prism 162 (FIGS. 6A to 6D, 9C), and loupe lens assembly 180 with loupe lens 182. In certain embodiments, loupe lens assembly 180 may be removably attached to housing 161 using one or more magnets, dovetail connections, etc. Generally, loupe lens 182 provides a fixed or variable wide field of view (or observation angle), magnification, etc. For example, different loupe lenses 182 may provide different wide fields of view generally between 60° and 180°, such as 60°, 90°, 120°, etc., or a range of wide fields of view, such as 60° to 120°, 60° to 130°, etc.

In certain embodiments, image inverter-reverter module 160 may be used without reduction lens assembly 150 to invert and revert the image viewed by the ophthalmic microscope.

Referring to FIG. 1A, reduction lens assembly 150 is depicted in the disengaged position and image inverter-reverter module 160 is depicted in the stowed position. An optical axis of image inverter-reverter module 160 is not aligned with the optical axis 11 of the ophthalmic microscope in the stowed position. Similarly, an optical axis 7 of reduction lens assembly 150 is not aligned with the optical axis 11 of the ophthalmic microscope in the disengaged position (FIG. 4A). In this configuration, the ophthalmic microscope may be used normally.

Referring to FIG. 1B, reduction lens assembly 150 is depicted in the disengaged position and image inverter-reverter module 160 is depicted in the partial stowed position. Image inverter-reverter module 160 has been rotated 90° about the 1^{st} axis 1 by the user from the stowed position to the partial stowed position. The optical axis of image inverter-reverter module 160 is not aligned with the optical axis 11 of the ophthalmic microscope in the partial stowed position. In this configuration, the ophthalmic microscope may be used normally.

Referring to FIG. 1C, reduction lens assembly 150 (without housing 141) is depicted in the engaged position and image inverter-reverter module 160 is depicted in the deployed position. In this configuration, the ophthalmic microscope may be used with WAVS 100 to provide a wide-angle field of view.

Image inverter-reverter module 160 has been rotated 90° about the 2^{nd} axis 2 by the user from the partial stowed position to the deployed position, while reduction lens assembly 150 has been translated along a 4^{th} axis 4 by the user from the disengaged position to the engaged position. The 4^{th} axis 4 is the axis along which reduction lens assembly 150 translates, and is parallel with the travel path of the slider 146 in the channel 148. In some embodiments, 4^{th} axis 4 may be generally parallel with the 1^{st} axis 1. The optical axis of image inverter-reverter module 160 is aligned with the optical axis 11 of the ophthalmic microscope when configured and arranged in the deployed position. Similarly, the optical axis 7 of reduction lens assembly 150 is aligned with the optical axis 11 of the ophthalmic microscope in the engaged position (FIG. 4C).

Slider 146 is attached to reduction lens assembly 150, and translates along channel 148 to allow the user to move reduction lens assembly 150 from the disengaged position to the engaged position (and vice versa) along the 4^{th} axis 4.

In certain embodiments, lever arm 144 may be coupled to reduction lens assembly 150 by gear train 145, which converts the rotation of lever arm 144 into the translation of reduction lens assembly 150 along the 4^{th} axis 4. Accordingly, lever arm 144 is also configured to allow the user to move reduction lens assembly 150 from the disengaged position to the engaged position (and vice versa). In some embodiments, reduction lens assembly 150 may also include slider 146. In some embodiments, reduction lens assembly 150 includes lever arm 144 in place of slider 146.

Image inverter-reverter module 160 may be translated by the user along the optical axis 11 of the ophthalmic microscope. In other words, when image inverter-reverter module 160 is disposed in the deployed position, the 3^{rd} axis 3 is parallel to the optical axis 11 of the ophthalmic microscope.

In certain embodiments, image inverter-reverter module 160 may include a proximity sensor (such as proximity sensor 610 depicted in FIG. 6C), located proximate to loupe lens assembly 180, to measure a distance between loupe lens 182 and the cornea. The proximity sensor may be an optical proximity sensor, an ultrasonic proximity sensor, etc., and may be coupled to the ophthalmic microscope by a signal cable.

In response to receiving the proximity sensor signal, the ophthalmic microscope may determine the distance between loupe lens 182 and the cornea. If the distance becomes less than a warning threshold distance (such as 2 inches, 3 inches, etc.), the ophthalmic microscope may provide a warning to the user, such as a graphical user interface (GUI) notification projected into an eyepiece of the ophthalmic microscope, an audible alarm, etc. If the distance becomes less than a danger threshold distance (such as 0.1 inches, 0.25 inches, 0.5 inches, etc.), the ophthalmic microscope may perform an automatic microscope focus move to translate at least a portion of the ophthalmic microscope and WAVS 100 up and away from the patient’s cornea.

In certain embodiments, WAVS 100 may include anti-fog or fog mitigation features within reduction lens module 140 and image inverter-reverter module 160, such as an anti-fog coating, a hydrophobic material, or any other technique to avoid or reduce fogging.

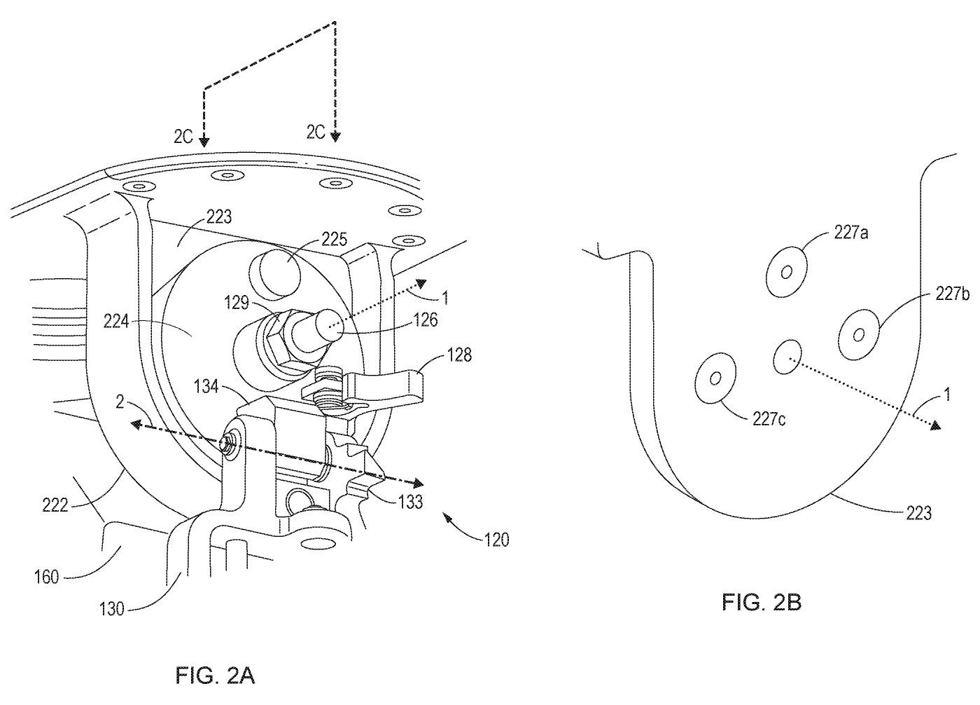

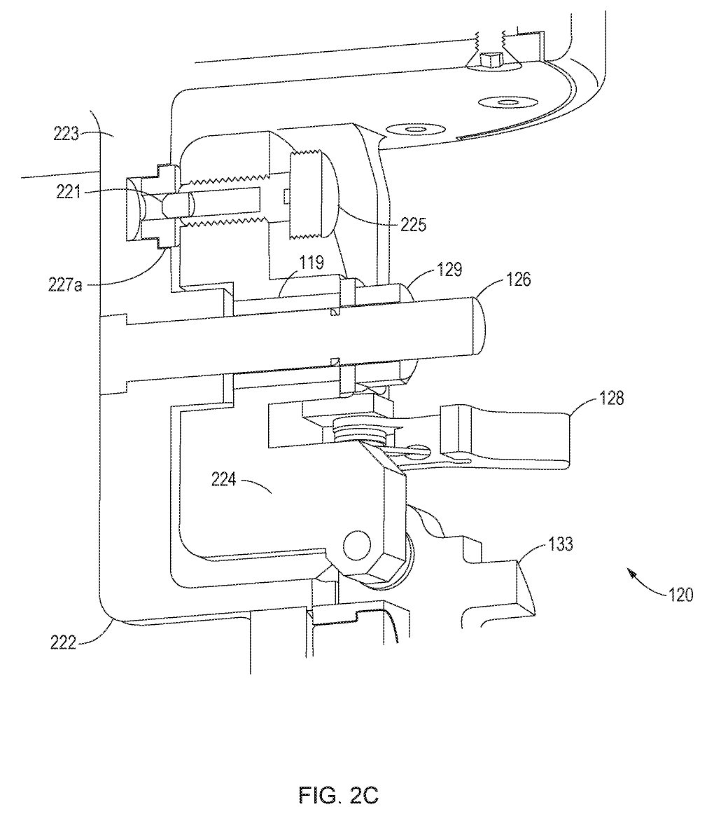

FIGS. 2A, 2B, and 2C depict a portion of frame 120 of WAVS 100, in accordance with embodiments of the present disclosure.

FIG. 2A depicts base 222 and portions of articulated support arm 130 and image inverter-reverter module 160 in the deployed position. Base 222 includes base support plate 223, rotation plate 224, spring plunger 225, bearing 119 (FIG. 2C), shaft 126, latch 128, and nut 129. Articulated support arm 130 includes bushing assembly 134 and pawl 133. In the stowed position or the partial stowed position, latch 128 engages pawl 133, while in the deployed position, latch 128 does not engage pawl 133. In certain embodiments, latch 128 may be spring loaded.

FIG. 2B depicts base support plate 223 including plunger seats 227a, 227b, 227c. Spring plunger 225 includes detent pin 221 (FIG. 2C) that engages plunger seat 227a when articulated support arm 130 and image inverter-reverter module 160 are disposed in the deployed position (or the partial stowed position). Conversely, detent pin 221 engages plunger seat 227b (or plunger seat 227c) when articulated support arm 130 and image inverter-reverter module 160 are disposed in the stowed position.

For example, to transition articulated support arm 130 and image inverter-reverter module 160 from the deployed position to the stowed position, spring plunger 225 is retracted to remove detent pin 221 from plunger seat 227a, articulated support arm 130 and image inverter-reverter module 160 are rotated +90° (or −90°) so that detent pin 221 engages plunger seat 227b (or plunger seat 227c). Or stated another way, WAVS 100 may include two distinct stowed configurations, one in which articulated support arm 130 and image inverter-reverter module 160 are rotated +90° from the deployed position, and another distinct stowed configuration in which articulated support arm 130 and image inverter-reverter module 160 are rotated -90° from the deployed position.

FIG. 2C depicts a sectional view of base 222 with articulated support arm 130 in the deployed position. Base support plate 223, rotating plate 224, spring plunger 225, detent pin 221, shaft 126, plunger seat 227a, latch 128, bearing 119, nut 129, and pawl 133 are shown.

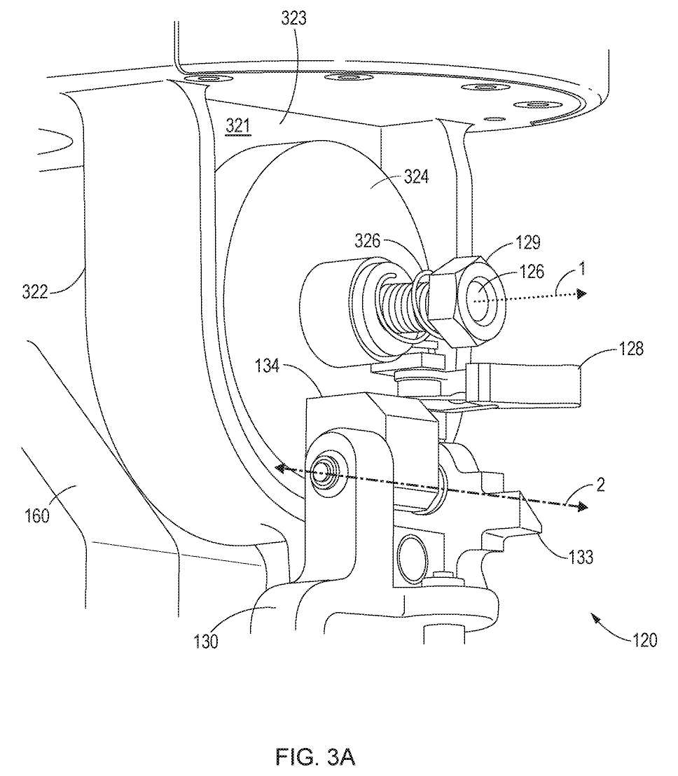

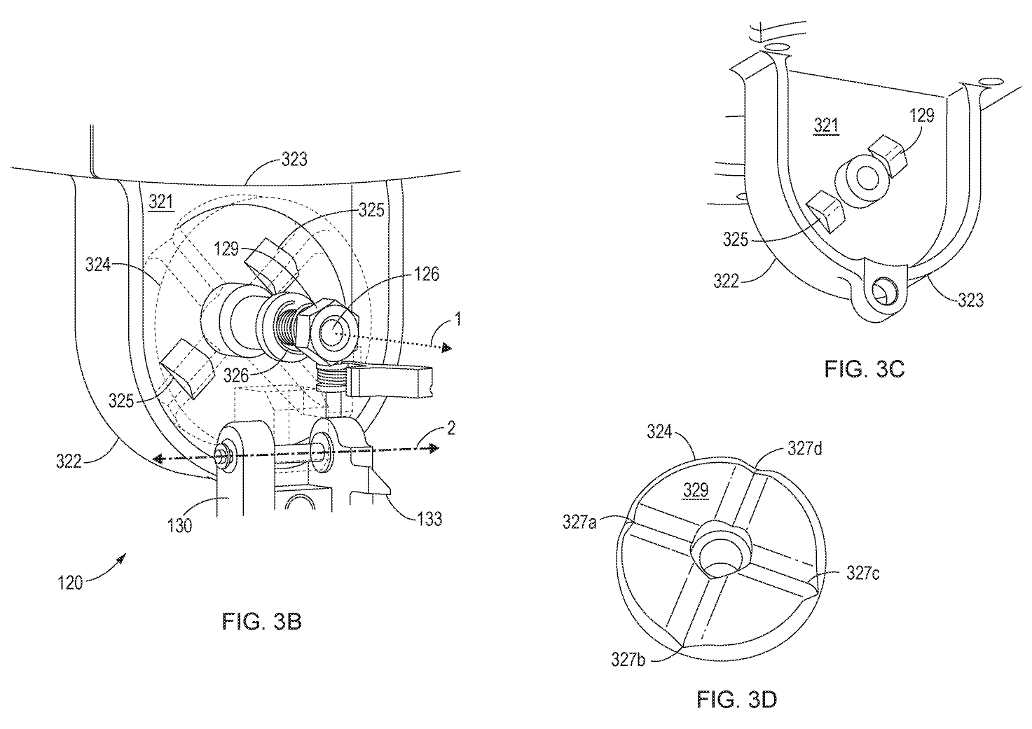

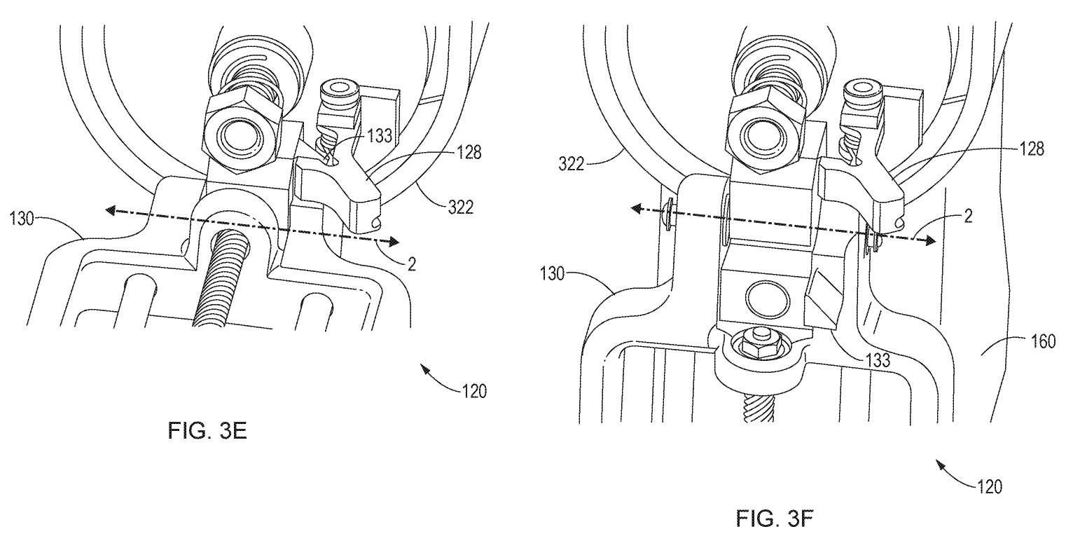

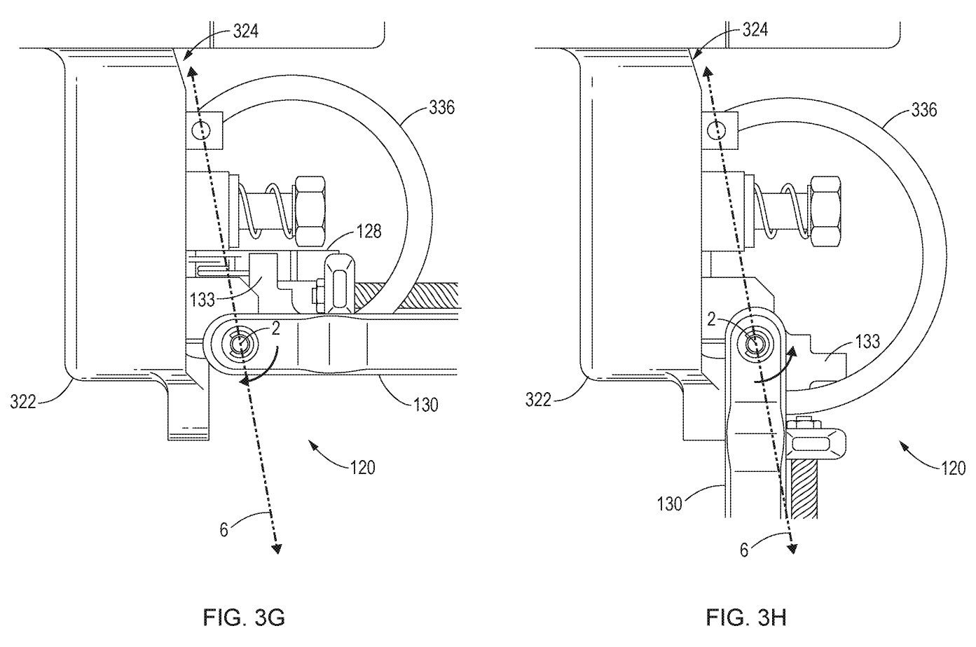

FIGS. 3A to 3H depict a portion of frame 120 of WAVS 100, in accordance with embodiments of the present disclosure.

FIG. 3A depicts base 322 and portions of articulated support arm 130 and image inverter-reverter module 160 in the deployed position. Base 322 includes base support plate 323, rotation plate 324, spring 326, shaft 126, latch 128, and nut 129. Articulated support arm 130 includes pawl 133 and bushing assembly 134. Because shaft 126 is attached to base support plate 323 and nut 129 is attached to shaft 126, spring 326 provides a compressive force against rotation plate 324. In other words, spring 326 pushes rotation plate 324 against base support plate 323 to maintain contact between reverse surface 329 of rotation plate 324 (FIG. 3D) and top surface 321 of base support plate 323.

FIG. 3B depicts base 322 (with rotation plate 324 shown in phantom as indicated by the dashed lines) and portions of articulated support arm 130 (with bushing assembly 134 shown in phantom as indicated by the dashed lines) in the deployed position. FIG. 3C depicts base 322, and FIG. 3D depicts reverse surface 329 of rotating plate 324.

Base support plate 323 includes two positive detents 325 extending above top surface 321, while rotating plate 324 includes four negative detents 327 recessed into reverse surface 329. Positive detents 325 may be spaced at 180°, and negative detents 327 may be spaced at 90° intervals to form two pairs of opposing negative detents 327 (327a and 327b, 327c and 327c). Other numbers and spacings for positive detents 325 and negative detents 327 are also supported.

Generally, spring 326 holds rotation plate 324 against base support plate 323, while positive detents 325 and the two pairs of opposing negative detents 327 (327a and 327c, 327b and 327d) cooperate to align rotation plate 324 in the deployed position (0°) or the stowed position (+90° or −90°).

For example, to transition articulated support arm 130 and image inverter-reverter module 160 from the deployed position to the stowed position, rotation plate 324 is rotated about the 1^{st} axis 1, which causes the pair of opposing negative detents 327a and 327c that are engaged with positive detents 325 to disengage (via a camming action of the negative detents 327 and the positive detents 325), which moves rotation plate 324 along the 1^{st} axis 1 toward nut 129 and further compresses spring 326. As rotation plate 324 approaches the stowed position, the other pair of opposing negative detents 327b and 327d engage positive detents 325. After rotating +90º (or −90°), the other pair of opposing negative detents 327b and 327d fully engage the two positive detents 325 to hold rotation plate 324, articulated support arm 130, and image inverter-reverter module 160 in the stowed position.

FIGS. 3E and 3F depict articulated support arm 130 and image inverter-reverter module 160 in the partial stowed position and the deployed position, respectively.

In the partial stowed position (and the stowed position), latch 128 engages pawl 133. In the deployed position, latch 128 does not engage pawl 133. In certain embodiments, latch 128 may be spring loaded.

FIGS. 3G and 3H depict articulated support arm 130 in the partial stowed position and the deployed position, respectively.

Spring 336 is attached to rotation plate 324 and articulated support arm 130. Generally, spring 336 assists the rotation of articulated support arm 130 from the partial stowed position to the deployed position (and vice versa). Spring 336 may be a C-spring, an extension spring, etc.

In the partial stowed position, latch 128 engages pawl 133, and spring 336 provides a torque about the 3^{rd} axis 3 that maintains articulated support arm 130 and image inverter-reverter module 160 in the partial stowed position. In the deployed position, latch 128 does not engage pawl 133, and spring 336 provides a torque about the 2^{nd} axis 2 (perpendicular to the view) that maintains articulated support arm 130 and image inverter-reverter module 160 in the deployed position.

For example, to transition articulated support arm 130 and image inverter-reverter module 160 from the partial stowed position to the deployed position, articulated support arm 130 is rotated about the 2^{nd} axis 2. Spring 336 resists this rotation until inflection axis 6 is reached, then spring 336 assists the final rotation of articulated support arm 130 into the deployed position. Similarly, to transition articulated support arm 130 and image inverter-reverter module 160 from the deployed position to the partial stowed position, articulated support arm 130 is rotated about the 2^{nd} axis 2. Spring 336 resists this rotation until inflection axis 6 is reached, then spring 336 assists the final rotation of articulated support arm 130 into the partial stowed position.

In another embodiment, a compression spring and cam mechanism may be positioned along the 2^{nd} axis 2, such that frame 120 will tend toward either the stowed position or the engaged position using the same inflection axis 6 as shown in FIG. 3G. The compression spring will be at maximum compression at the inflection point.

FIGS. 4A and 4B depict an upper portion of WAVS 100, in accordance with embodiments of the present disclosure.

FIG. 4A depicts reduction lens module 140 with reduction lens assembly 150 in the disengaged position. As discussed above, the optical axis 7 of reduction lens assembly 150 is not aligned with the optical axis 11 of the ophthalmic microscope in the disengaged position. Mount 142, lever arm 144, gear train 145, slider 146, and channel 148 of reduction lens module 140, as well as base 122 and shaft 126 of frame 120, are also depicted. Housing 141 is depicted in phantom as indicated by the dashed lines.

FIG. 4B depicts reduction lens module 140 with reduction lens assembly 150 in a partially engaged position. Here, reduction lens assembly 150 has been translated along the 4^{th} axis 4 by the user from the disengaged position to the partially engaged position by moving slider 146 and/or rotating lever arm 144. Mount 142, gear train 145, and channel 148 of reduction lens module 140, as well as base 122 and shaft 126 of frame 120, are also depicted. Housing 141 is depicted in phantom as indicated by the dashed lines.

FIG. 4C depicts a sectional view of reduction lens module 140 with reduction lens assembly 150 in the engaged position, in accordance with embodiments of the present disclosure.

As discussed above, the optical axis 7 of reduction lens assembly 150 is aligned with the optical axis 11 of the ophthalmic microscope in the engaged position. Reduction lens assembly 150 includes housing 151, adjustable reduction lens 152, and reduction lens adjustment mechanism (not shown for clarity). In certain embodiments, adjustable reduction lens 152 may be configured to translate along optical axis 7 of reduction lens assembly 150. In other embodiments, reduction lens assembly 150 may be configured to translate along its optical axis 7. Reduction lens adjustment mechanism may include a mechanical linkage operated by the user, an electric motor operated by a switch or button, etc. For example, an electric motor and linear guide may be located within chamber 441. Generally, adjustable reduction lens 152 may reduce the focal length and adjust the focus of the ophthalmic microscope and WAVS 100 optical system.

In certain embodiments, adjustable reduction lens 152 may be a doublet lens that includes reduction lens 451, reduction lens 452, and lens mount 153. Reduction lens adjustment mechanism may be configured to translate reduction lens 451, reduction lens 452, and lens mount 153 along optical axis 7 of reduction lens assembly 150 as a single component. Alternatively, lens mount 153 may include a fixed upper portion and a movable lower portion, reduction lens 451 may be attached to the fixed upper portion of lens mount 153, reduction lens 452 may be attached to the movable lower portion of lens mount 153, and reduction lens adjustment mechanism may be configured to translate reduction lens 452 and the movable portion of lens mount 153 along optical axis 7 of reduction lens assembly 150 as a single component. In other embodiments, adjustable reduction lens 152 may be a singlet lens, a triplet lens, etc.

Housing 141, mount 142, cam lever 143, lever arm 144, and gear train 145 of reduction lens module 140, bearing 147, shaft 149, locking pin 453, slot 454 and channel 455 of reduction lens assembly 150, and base 122 of frame 120, are also depicted in FIG. 4C.

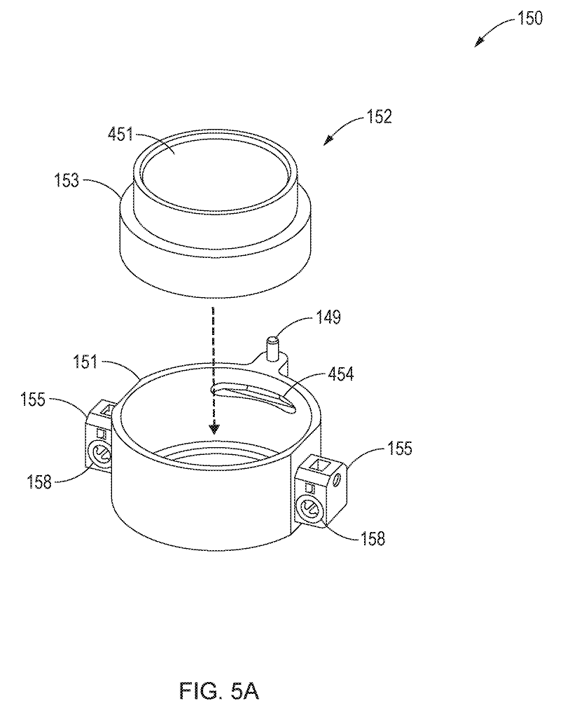

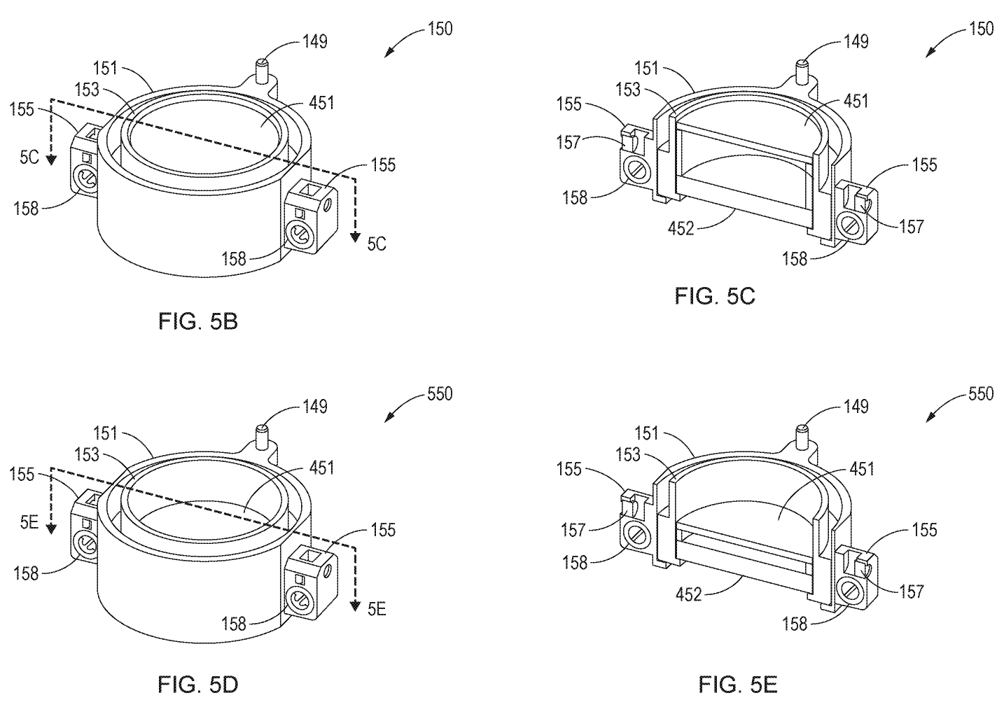

Locking pin 453 is inserted into slot 454 to secure adjustable reduction lens 152 within housing 151 of reduction lens assembly 150 via a press fit, a friction fit, etc. Adjustable reduction lens 152 is removable, and may be replaced with different lens cells, as described with respect to FIGS. 5A to 5E. Shaft 149 is disposed within channel 455 to support bearing 147.

FIGS. 4D, 4E, and 4F depict views of reduction lens module 140 with housing 141 removed, in accordance with embodiments of the present disclosure

FIG. 4D depicts reduction lens module 140 with reduction lens assembly 150 in the disengaged position. Cam lever 143 is attached to gear train 145, and rotation of lever arm 144 is transferred by gear train 145 into rotation of cam lever 143. Bearing 147 translates the rotation of cam lever 143 into linear translation of reduction lens assembly 150 by moving along an inner cam surface of cam lever 143.

FIG. 4E depicts reduction lens module 140 with reduction lens assembly 150 in a partial engaged position. Lever arm 144 has been rotated counter-clockwise from the disengaged position, which translates reduction lens assembly 150 to the partial engaged position. Cam lever 143 and bearing 147 are also identified.

FIG. 4F depicts reduction lens module 140 with reduction lens assembly 150 in the engaged position. As discussed above, the optical axis 7 of reduction lens assembly 150 is aligned with the optical axis 11 of the ophthalmic microscope in the engaged position. Lever arm 144 has been rotated counter-clockwise from the partial engaged position, which translates reduction lens assembly 150 to the engaged position. Cam lever 143, bearing 147, and shafts 156 (FIGS. 4H, 4I) are also identified.

FIG. 4G depicts reduction lens module 140 with reduction lens assembly 150 in the engaged position, in accordance with embodiments of the present disclosure. Sliders 146 are identified on each side of reduction lens assembly 150, while channel 148 is identified on one side of reduction lens assembly 150.

FIG. 4H depicts a sectional view of reduction lens module 140 with reduction lens assembly 150 in the engaged position, while FIG. 4I depicts a portion of FIG. 4H identified by the dotted circle, in accordance with embodiments of the present disclosure. As discussed above, the optical axis 7 of reduction lens assembly 150 is aligned with the optical axis 11 of the ophthalmic microscope in the engaged position.

Sliders 146 and channels 148 are depicted on each side of reduction lens module 140. Reduction lens assembly 150 translates along shafts 156, while magnets 157, 159 magnetically couple sliders 146 to reduction lens assembly 150. Accordingly, translation of sliders 146 within channels 148 also translates reduction lens assembly 150 along shafts 156, which moves reduction lens assembly 150 from the disengaged position to the engaged position (and vice versa). Bearings 158 may slidingly couple reduction lens assembly 150 to shafts 156.

FIGS. 5A to 5E depict reduction lens assembly 150, in accordance with embodiments of the present disclosure.

FIG. 5A depicts an exploded view of reduction lens assembly 150. Lens mount 153 and reduction lens 451 of adjustable reduction lens 152, as well as shaft 149, slot 454, arms 155, and bearings 158 of housing 151 are identified.

FIG. 5B depicts reduction lens assembly 150. Lens mount 153 and reduction lens 451 of adjustable reduction lens 152, as well as shaft 149, arms 155, and bearings 158 of housing 151 are identified.

FIG. 5C depicts a sectional view of reduction lens assembly 150. Lens mount 153, reduction lens 451 and reduction lens 452 of adjustable reduction lens 152, as well as shaft 149, arms 155, magnets 157, and bearings 158 of housing 151 are identified. FIGS. 5A, 5B, and 5C depict an embodiment of adjustable reduction lens 152 that provides a working distance of 175 millimeters (mm).

FIG. 5D depicts reduction lens assembly 550. Lens mount 153 and reduction lens 451 of adjustable reduction lens 152, as well as shaft 149, arms 155, and bearings 158 of housing 151 are identified.

FIG. 5E depicts a sectional view of reduction lens assembly 550. Lens mount 153, reduction lens 451 and reduction lens 452 of adjustable reduction lens 152, as well as shaft 149, arms 155, magnets 157, and bearings 158 of housing 151 are identified. FIGS. 5D and 5E depict an embodiment of adjustable reduction lens 550 that provides a working distance of 200 mm.

Either reduction lens assembly 150 or reduction lens assembly 550 may be installed in reduction lens module 140.

FIGS. 6A, 6B, and 6C depict a lower portion of WAVS 100, in accordance with embodiments of the present disclosure. An optical axis 8 of image inverter-reverter module 160 is depicted.

Articulated support arm 130 and image inverter-reverter module 160 are depicted in the deployed position. As discussed above, the optical axis 8 of image inverter-reverter module 160 is aligned with the optical axis 11 of the ophthalmic microscope in the deployed position.

Image inverter-reverter module 160 includes housing 161 (in partial cutaway), optical prism 162, upper protective window 170, support mount 164, support mount adjustment screws 165, and lower protective window 172. Upper protective window 170 and lower protective window 172 may be optically transparent material. Generally, optical prism 162 may be a direct vision, reflective optical prism that both inverts and reverts the image viewed by the ophthalmic microscope and WAVS 100 optical system, such as an Abbe Koenig prism, a Porro prism, a Dove prism, etc., a combination of an inverter prism and a reverter prism, etc.

Articulated support arm 130 includes body 131, hinge 132, bushing assembly 134 (FIG. 1B, 1C), threaded shaft 136, guide rods 137, cradle 138, and knob 139. Cradle 138 is attached to support mount 164 using support mount adjustment screws 165 which allow image inverter-reverter module 160 to be precisely aligned to the optical axis 11 of the ophthalmic microscope in the deployed position. In certain embodiments, three support mount adjustment screws 165 may be provided on each side of support mount 164; other types of alignment mechanisms are also supported.

For example, one alignment method includes placing WAVS 100 in a fixture to align the optical axes of reduction lens assembly 150, image inverter-reverter module 160 and loupe lens assembly 180, and injecting epoxy into the gaps (not visible) in support mount 164, right-angle support arm 184, and planar support arm 186. Alternatively, support mount adjustment screws 165 may lock the location of image inverter-reverter module 160 while in the fixture. The rotation adjustment ensures the axes do not drift during vertical motion. The tolerances of the openings surrounding each screw 165 allow for translation and rotation motion along the orthogonal axes to lock the location of image inverter-reverter module 160 while in the fixture.

FIGS. 6A and 6C depict cradle 138 disposed in a first position (or upper position), while FIG. 6B depicts cradle 138 disposed in a second position (or intermediate position). As discussed above, knob 139 rotates threaded shaft 136, which translates cradle 138 (and image inverter-reverter module 160) along the 3^{rd} axis 3 over the length of body 131, such as between the first position and the second position.

Loupe lens assembly mount 166 is configured to receive loupe lens assembly 180 with right-angle support arm 184, while loupe lens assembly mount 163 is configured to receive loupe lens assembly 180 with planar support arm 186. Image inverter-reverter module 160 may include loupe lens assembly mount 166, loupe lens assembly mount 163, or both loupe lens assembly mount 166 and 163. Loupe lens assembly 180 may be removably attached to loupe lens assembly mount 166 or 163 using one or more magnets, dovetail connections, etc. In certain embodiments, proximity sensor 610 may be attached to support mount 164 to measure the distance between loupe lens 182 and the patient’s cornea. The proximity sensor may be an optical proximity sensor, an ultrasonic proximity sensor, etc., and may be coupled to the ophthalmic microscope by a signal cable.

FIG. 6D depicts cradle 138 disposed in the second position, while FIG. 6E depicts a first portion of cradle 138 disposed in the first position and a second portion of cradle 138 disposed in the second position, in accordance with embodiments of the present disclosure. FIG. 6F depicts a cross-sectional view of a portion of FIG. 6D identified by the dotted circle, while FIG. 6G depicts a cross-sectional view of a portion of FIG. 6E identified by the dotted circle.

In certain embodiments, cradle 138 may include cradle portion 601 and cradle portion 602. Cradle portion 601 is attached to image inverter-reverter module 160 and includes magnet 604. Cradle portion 602 includes magnet 604 and threaded portion 603 through which threaded shaft 136 passes. Cradle portion 601 and cradle portion 602 are magnetically coupled during normal operation, as depicted in FIG. 6D. As discussed above, knob 139 rotates threaded shaft 136, which translates cradle portion 601 (and image inverter-reverter module 160) and cradle portion 602 along the 3^{rd} axis 3 over the length of body 131, such as between the first position and the second position (and vice versa).

Advantageously, image inverter-reverter module 160 may be moved away from the patient’s eye without rotating knob 139 by simply breaking the magnetic coupling between magnets 604 and moving cradle portion 601 (and image inverter-reverter module 160) along guide rods 137 from the second position to the first position.

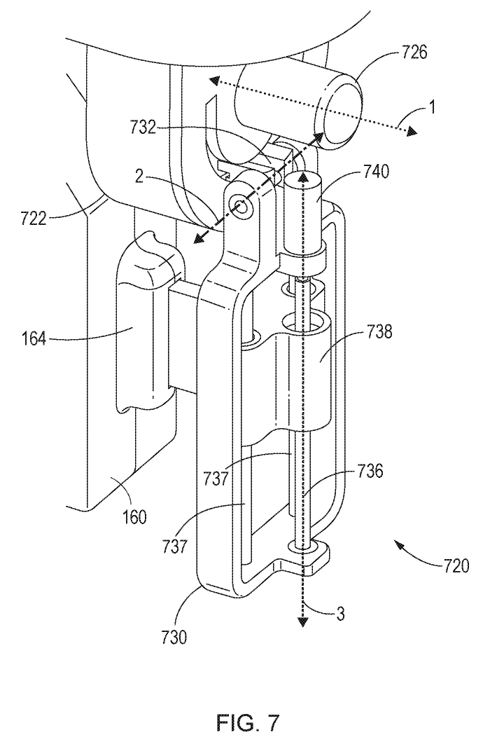

FIG. 7 depicts the lower portion of WAVS 100, in accordance with embodiments of the present disclosure. In particular, FIG. 7 depicts a frame 720 with motors (such as motors 726 and 740) to facilitate movement of the frame 720 or components attached thereto in an automated manner. Frame 720 can be used in place of frame 120 described above.

In certain embodiments, frame 720 includes base 722 and motorized support arm 730. Base 722 includes motor 726 which has an output shaft that defines the 1^{st} axis 1 for rotation of motorized support arm 730 and image inverter-reverter module 160. As described above, the 1^{st} axis 1 is perpendicular to the optical axis 11 of the ophthalmic microscope when WAVS 100 is installed. More particularly, motor 726 rotates motorized support arm 730 (and image inverter-reverter module 160) about the 1^{st} axis 1, from the stowed position (+90° or −90°) to the deployed position (0°) (and vice versa). Base 722 may be attached to reduction lens module 140 using fasteners (such as bolts, screws, etc.), soldering, welding, etc.

Motorized support arm 730 is coupled to base 722 such that it can be moved and/or rotated relative to base 722. Motorized support arm 730 facilitates movement of the image inverter-reverter module 160 about the motorized hinge 732 via a motor (not shown) and along the threaded shaft 736 via motor 740. Motorized support arm 730 includes motorized hinge 732, threaded shaft 736, guide rods 737, cradle 738, and motor 740.

Motorized hinge 732 defines the 2^{nd} axis 2, and includes a motor (not shown) to rotate motorized support arm 730 (and image inverter-reverter module 160) about the 2^{nd} axis 2 from the partial stowed position to the deployed position (and vice versa).

Motor 740 is coupled to threaded shaft 736, which defines the 3^{rd} axis 3. Motor 740 rotates threaded shaft 736 which translates cradle 738 (and image inverter-reverter module 160) along the 3^{rd} axis 3 over the length of body 131, similar to knob 139 described above. Motorized support arm 730 and image inverter-reverter module 160 are depicted in the deployed position.

Motor 726, the motor (not shown) of motorized hinge 732, and motor 740 may be controlled using switches or buttons, etc. In certain embodiments, motor 726, the motor of motorized hinge 732, and motor 740 may be connected to a computer and controlled using a computer interface device, such as a mouse, a touchpad, a keyboard, a foot pedal, etc.

Cradle 738 may be attached to support mount 164 using support mount adjustment screws (such as support mount adjustment screws 165) which allow image inverter-reverter

module 160 to be precisely aligned to the optical axis 11 of the ophthalmic microscope in the deployed position.

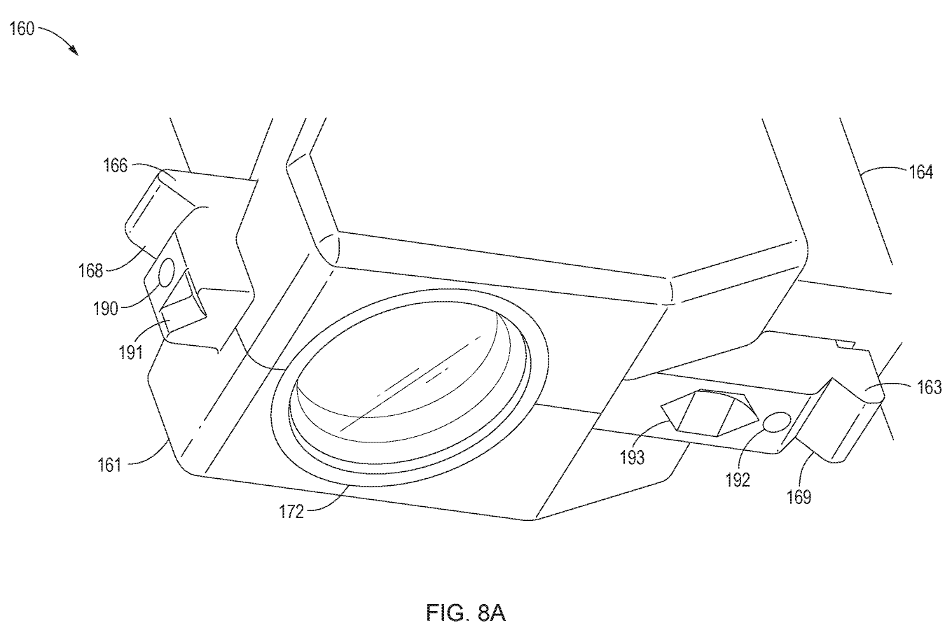

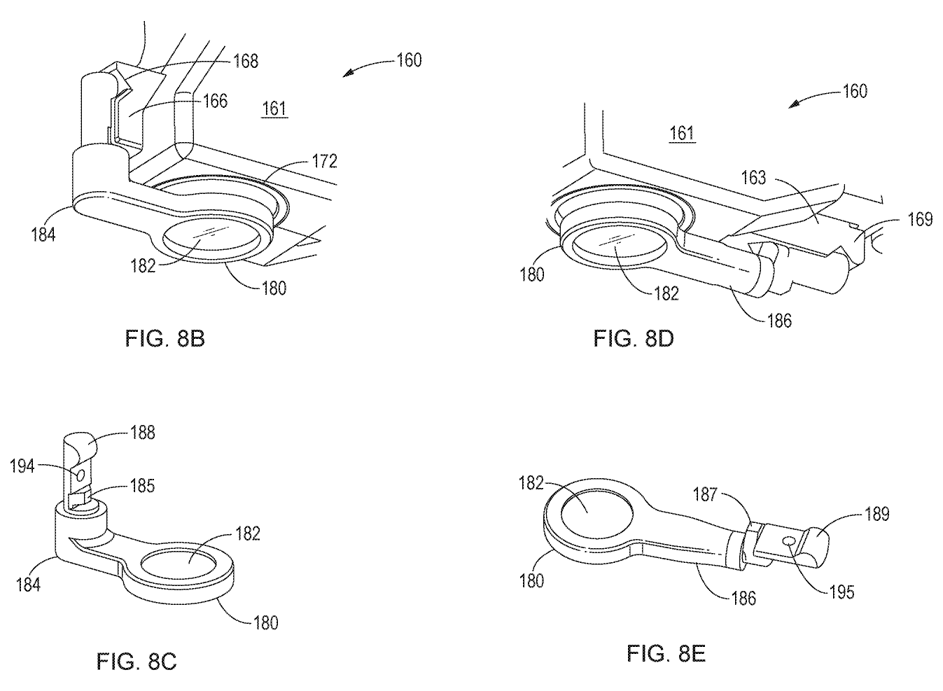

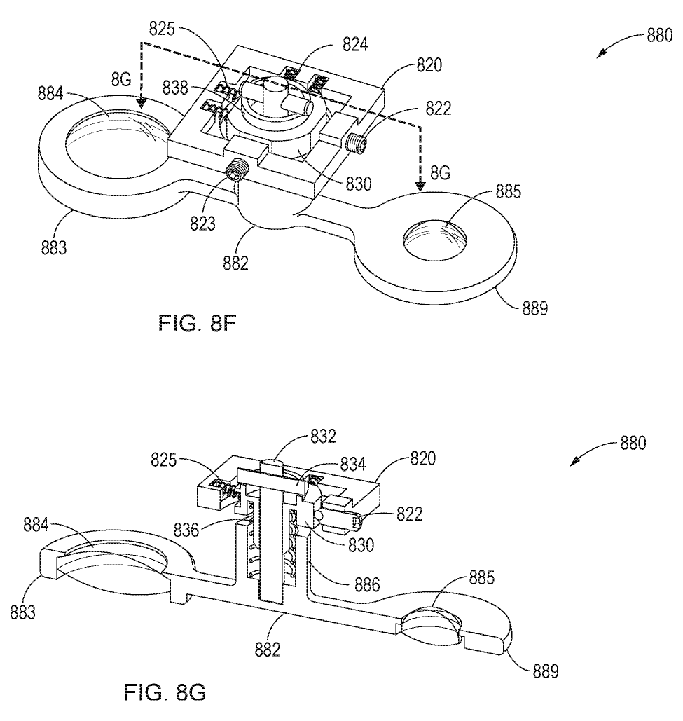

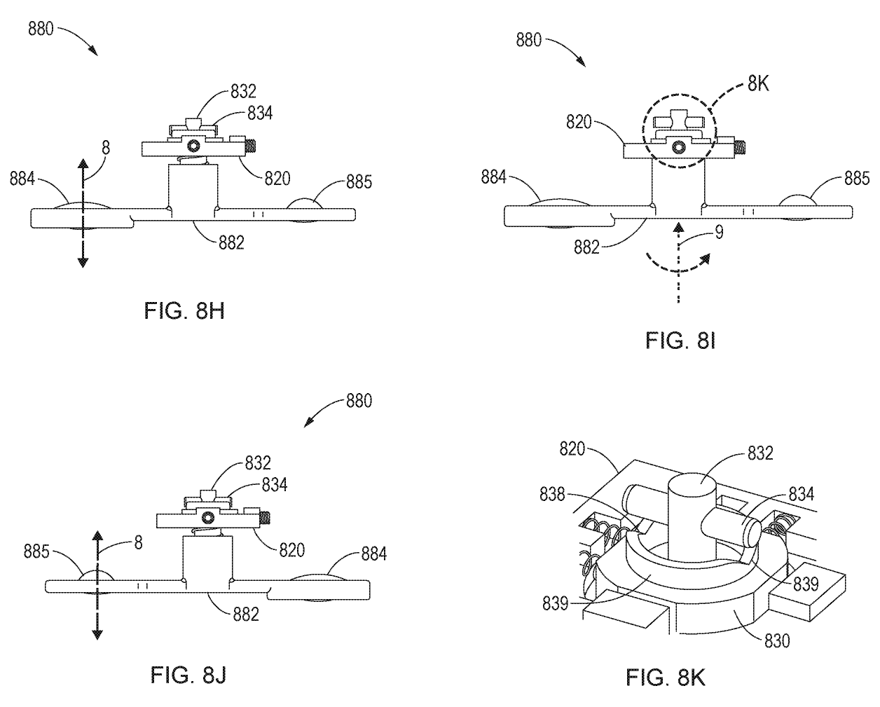

FIG. 8A depicts a lower portion of image inverter-reverter module 160, in accordance with embodiments of the present disclosure.

More particularly, housing 161, support mount 164, loupe lens assembly mounts 166 and 163, and lower protective window 172 are depicted. Loupe lens assembly mount 166 includes v-recess 191, v-channel 168, and magnet 190. Similarly, loupe lens assembly mount 163 includes v-recess 193, v-channel 169, and magnet 192. Generally, loupe lens assembly mount 166 supports loupe lens assembly 180 with right-angle support arm 184 (for example, as illustrated in FIGS. 8B and 8C), while loupe lens assembly mount 163 supports loupe lens assembly 180 with planar support arm 186 (for example, as illustrated in FIGS. 8D and 8E).

FIG. 8B depicts the lower portion of image inverter-reverter module 160, in accordance with embodiments of the present disclosure. More particularly, housing 161, loupe lens assembly mount 166, lower protective window 172, and loupe lens assembly 180 with loupe lens 182 and right-angle support arm 184, are depicted.

FIG. 8C depicts loupe lens assembly 180 with loupe lens 182 and right-angle support arm 184, in accordance with embodiments of the present disclosure.

In certain embodiments, loupe lens assembly 180 also includes v-protrusion 185, v-protrusion 188, and magnet 194. V-recess 191 is configured to receive v-protrusion 185, v-recess 168 is configured to receive v-protrusion 188, and magnets 190 and 194 cooperate to magnetically coupled loupe lens assembly 180 to loupe lens assembly mount 166. Generally, v-recess 191 and v-protrusion 185 provide stability in a first direction (such as a longitudinal direction), while v-recess 168 and v-protrusion 188 provide stability in a second direction perpendicular to the first direction (such as a transverse direction).

FIG. 8D depicts the lower portion of image inverter-reverter module 160, in accordance with embodiments of the present disclosure. More particularly, housing 161, loupe lens assembly mount 163, lower protective window 172, and loupe lens assembly 180 with loupe lens 182 and planar support arm 186, are depicted.

FIG. 8E depicts loupe lens assembly 180 with loupe lens 182 and planar support arm 186, in accordance with embodiments of the present disclosure.

In certain embodiments, loupe lens assembly 180 also includes v-protrusion 187, v-protrusion 189, and magnet 195. V-recess 193 is configured to receive v-protrusion 187, v-recess 169 is configured to receive v-protrusion 189, and magnets 192 and 195 cooperate to magnetically coupled loupe lens assembly 180 to loupe lens assembly mount 163. Generally, v-recess 193 and v-protrusion 187 provide stability in a first direction (such as a first transverse direction), while v-recess 169 and v-protrusion 189 provide stability in a second direction perpendicular to the first direction (such as a second transverse direction).

FIG. 8F depicts loupe lens assembly 880, while FIG. 8G depicts a sectional view of loupe lens assembly 880, in accordance with embodiments of the present disclosure.

Generally, loupe lens assembly 880 may be mounted to image inverter-reverter module 160 in place of loupe lens assembly mount 163, and rotates to place either loupe lens 884 or loupe lens 885 in the optical axis of image inverter-reverter module 160.

In certain embodiments, loupe lens assembly 880 includes outer frame 820 mounted to image inverter-reverter module 160, adjustment screw 822, at least one adjustment spring 824, adjustment screw 823, at least one adjustment spring 825, inner frame 830 including shoulder 838, central shaft 832, transverse shaft 834, spring 836, and loupe lens arm 882 including loupe lens 884 and loupe lens 885.

Loupe lens arm 882 includes body 886 defining central passage 887, first arm 883 extending from body 886, and second arm 889 extending from body 886. Central passage 887 is configured to receive a portion of inner frame 830, central shaft 832, and spring 836. Central shaft 832 is slidingly coupled to inner frame 830, and is attached to a central portion of loupe lens arm 882. Spring 836 abuts the central portion of loupe lens arm 884 and a lower surface of inner frame 830. Loupe lens 884 is attached to an end of first arm 883, while loupe lens 885 is attached to an end of second arm 889.

Transverse shaft 834 is attached to central shaft 832. Transverse shaft 834 extends from central shaft 832 and abuts two opposing grooves 839 (FIG. 8H) in shoulder 838 of inner frame 830. Due to the arrangement of spring 836, transverse shaft 834 is held against grooves 839 of shoulder 838 in a secured position (depicted in FIGS. 8F, 8G, 8H, 8J).

Adjustment screw 823 cooperates with adjustment spring 824 to adjust the position of inner frame 830 with respect to outer frame 820 in a first transverse direction. Similarly, adjustment screw 822 cooperates with adjustment spring 825 to adjust the position of inner frame 830 with respect to outer frame 820 in a second transverse direction that is perpendicular to the first transverse direction. Adjusting the position of inner frame 830 with respect to outer frame 820 aligns loupe lens 884 or loupe lens 885 with the optical axis of image inverter-reverter module 160.

FIGS. 8H, 8I, 8J, 8K depict loupe lens assembly 880 in various positions, in accordance with embodiments of the present disclosure.

FIG. 8H depicts loupe lens assembly 880 in the secured position, with loupe lens 884 aligned in the optical axis 8 of image inverter-reverter module 160. Outer frame 820, central shaft 832, transverse shaft 834, loupe lens arm 882, and loupe lens 885 are also identified.

To place loupe lens 884 in the optical axis 8 of image inverter-reverter module 160, loupe lens arm 882 is first moved toward outer frame 820, which compresses spring 836 and raises transverse shaft 834 out of grooves 839 and above shoulder 838.

FIG. 8I depicts loupe lens assembly 880 in a transitional position, with transverse shaft 834 raised above shoulder 838 to allow loupe lens arm 882 to rotate about axis 9 freely. Outer frame 820, inner frame 830, central shaft 832, transverse shaft 834, shoulder 838, grooves 839, loupe lens arm 882, loupe lens 884, and loupe lens 885 are also identified. Loupe lens arm 882 is then rotated 180° to align loupe lens 885 in the optical axis 8 of image inverter-reverter module 160, and then loupe lens arm 882 is moved away from outer frame 820, which lowers transverse shaft 834 into grooves 839.

FIG. 8J depicts loupe lens assembly 880 in the secured position, with loupe lens 885 aligned in the optical axis 8 of image inverter-reverter module 160. Outer frame 820, central shaft 832, transverse shaft 834, loupe lens arm 882, and loupe lens 885 are also identified.

FIG. 8K depicts a perspective view of the portion of FIG. 8I identified by the dotted circle (labeled 8K in FIG. 8I).

FIG. 9A depicts ray trace diagram 900 for an ophthalmic microscope, in accordance with embodiments of the present disclosure.

Ray trace diagram 900 depicts optical axis 910, objective lens 920, and illumination module 930 of the ophthalmic microscope, and eye model 940 including cornea/lens 942 and retina 944. The focal plane lies on a surface of the cornea, and focal length (FL) 914 and working distance (WD) 916 are also depicted. Ray trace diagram 900 depicts the ophthalmic microscope operating in an alignment or focus mode, in which the optical components of WAVS 100 are not located within the optical path, i.e., adjustable reduction lens 152, optical prism 162, and loupe lens 182.

In certain embodiments, ophthalmic microscope may have a FL of 265mm and a WD of 200mm, while in other embodiments, ophthalmic microscope may have a FL of 240mm and a WD of 175mm.

FIG. 9B depicts ray trace diagram 902 for an ophthalmic microscope with WAVS 100, in accordance with embodiments of the present disclosure.

Ray trace diagram 900 depicts optical axis 910, objective lens 920, and illumination module 930 of the ophthalmic microscope, adjustable reduction lens 152, optical prism 162 and loupe lens 182 of WAVS 100, and eye model 940 including cornea/lens 942 and retina 944. Due to the effects of adjustable reduction lens 152, loupe lens 182, and cornea/lens 942, the focal plane has moved from the surface of the cornea to the surface of retina 944. Because the image of retina 944 is inverted and reverted when viewed through cornea/lens 942, optical prism 162 compensates for these effects by inverting and reverting the image of retina 944.

Advantageously, WD 916 does not change when the optical components of WAVS 100 are located within the optical path of the ophthalmic microscope, i.e., adjustable reduction lens 152, optical prism 162, and loupe lens 182.

FIG. 9C depicts optical prism 162 for image inverter-reverter module 160, in accordance with embodiments of the present disclosure.

In certain embodiments, optical prism 162 is a direct vision reflective prism, such as an Abbe-Koenig prism, etc. For purposes of description, the three principle axes of optical prism 162 are the longitudinal axis, the lateral axis, and the vertical axis. The longitudinal axis is parallel to optical axis 910, and the lateral and vertical axes are perpendicular to the longitudinal axis (and to one another).

Optical prism 162 may be formed by cementing two optical (glass) prisms 960, 962 together. Optical prism 960 has 8 surfaces or faces, while optical prism 962 has 6 surfaces or faces. Optical prism 960 includes incident face 970, lower face 972, upper (roof) faces 974, 975, inclined faces 976, 977, and lateral faces 978, 979. Optical prism 962 includes incident face 980, lower face 982, upper face 984, inclined face 986, and lateral faces 988, 989. A portion of lower face 972 may be cemented to upper face 984 to form optical prism 962. In other embodiments, optical prism 962 may be formed from a single piece of optical glass.

Incident faces 970, 980 are parallel to the vertical axis and perpendicular to the longitudinal axis (i.e., the normal of each incident face 970, 980 is parallel to the longitudinal axis). Lower faces 972, 982 are parallel to the lateral axis and inclined at a 30° angle with respect to the longitudinal axis. Upper surfaces 974, 975 are parallel to the longitudinal axis and inclined at a 57° angle with respect to the vertical axis.

For light rays traveling from left to right, the light rays enter and pass through incident face 970 (normal or 90° incident angle), strike lower face 972 (30° incident angle) and are reflected upwards, strike upper face 974 (or 975) and are reflected laterally, strike upper face 975 (or 974) and are reflected downwards, strike lower surface 982 (30° incident angle) and are reflected longitudinally, and exit incident face 980 (normal or 90° incident angle). Due to the inverting and reverting effects of optical prism 962, light rays that enter incident face 970 on the left (lateral side) of optical axis 910 exit incident face 980 on the right (lateral side) of optical axis 910, while light rays that enter incident face 970 above optical axis 910 exit incident face 980 below optical axis 910. Similarly, light rays that enter incident face 970 on the right (lateral side) of optical axis 910 exit incident face 980 on the left (lateral side) of optical axis 910, while light rays that enter incident face 970 below optical axis 910 exit incident face 980 above optical axis 910. Light rays traveling from the right to the left behave in a similar manner.

For example, light provided by illumination module 930 may pass from left to right, while light reflected from retina 944 (i.e., the image of retina 944) may pass from right to left.

In certain embodiments, an ophthalmic visualization system may include an ophthalmic microscope with an integrated WAVS 100. For example, WAVS 100 may be integrated into an extended body or housing of the ophthalmic microscope.

The certain features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. An apparatus for an ophthalmic microscope, comprising:
a frame;
a reduction lens module attached to the frame, the reduction lens module including:
a mount configured to be attached to an ophthalmic microscope, and
a movable reduction lens assembly including an adjustable reduction lens, the movable reduction lens assembly having an optical axis, a disengaged position, and an engaged position; and
an image inverter-reverter module movably coupled to the frame, the image inverter-reverter module including an optical prism and a loupe lens assembly, the image inverter-reverter module having an optical axis, a stowed position, and a deployed position,
wherein, when the mount is attached to the ophthalmic microscope and the movable reduction lens assembly is disposed in the engaged position, the optical axis of the movable reduction lens assembly is aligned with an optical axis of the ophthalmic microscope, and
wherein, when the mount is attached to the ophthalmic microscope and the image inverter-reverter module is disposed in the deployed position, the optical axis of the image inverter-reverter module is aligned with the optical axis of the ophthalmic microscope.

2. The apparatus of claim 1, wherein the mount is a bayonet mount that is configured to be attached to an illumination module of the ophthalmic microscope.

3. The apparatus of claim 1, wherein the adjustable reduction lens is configured to translate along the optical axis of the movable reduction lens assembly.

4. The apparatus of claim 3, wherein the adjustable reduction lens is a doublet lens.

5. The apparatus of claim 1, wherein:
the optical prism is an Abbe-Koenig prism;
the loupe lens assembly includes a lens having a wide field of view; and
the loupe lens assembly is removably coupled to a housing of the image inverter-reverter module.

6. The apparatus of claim 1, wherein the frame includes:
a base attached to the reduction lens module; and
an articulated support arm that is coupled to the image inverter-reverter module.

7. The apparatus of claim 6, wherein the articulated support arm is configured to rotate about a first axis that is perpendicular to the optical axis of the ophthalmic microscope.

8. The apparatus of claim 7, wherein the articulated support arm is configured to rotate about a second axis that is perpendicular to the first axis.

9. The apparatus of claim 8, wherein:
the articulated support arm includes a movable cradle that is attached to the image inverter-reverter module;
the movable cradle is configured to translate along a third axis that is perpendicular to the second axis; and
the third axis is parallel to the optical axis of the image inverter-reverter module.

10. The apparatus of claim 9, wherein, when the image inverter-reverter module is disposed in the deployed position, the third axis is parallel to the optical axis of the ophthalmic microscope.

11. The apparatus of claim 1, wherein, when the movable reduction lens assembly is disposed in the disengaged position, the optical axis of the movable reduction lens assembly is not aligned with the optical axis of the ophthalmic microscope.

12. The apparatus of claim 1, wherein, when the image inverter-reverter module is disposed in the stowed position, the optical axis of the image inverter-reverter module is not aligned with the optical axis of the ophthalmic microscope.

13. An ophthalmic visualization system, comprising:
an ophthalmic microscope having an optical axis;
a reduction lens module attached to the ophthalmic microscope, the reduction lens module including:
a movable reduction lens assembly including an adjustable reduction lens; and
an image inverter-reverter module movably coupled to the reduction lens module, the image inverter-reverter module including:
an optical prism, and
a loupe lens assembly,
wherein the movable reduction lens assembly has an optical axis, a disengaged position, and an engaged position, and, when the movable reduction lens assembly is disposed in the engaged position, the optical axis of the movable reduction lens assembly is aligned with the optical axis of the ophthalmic microscope, and
wherein the image inverter-reverter module has an optical axis, a stowed position, and a deployed position, and, when the image inverter-reverter module is disposed in the deployed position, the optical axis of the image inverter-reverter module is aligned with the optical axis of the ophthalmic microscope.

14. The ophthalmic visualization system of claim 13, wherein the adjustable reduction lens is configured to translate along the optical axis of the movable reduction lens assembly.

15. The ophthalmic visualization system of claim 14, wherein the adjustable reduction lens is a doublet lens.

16. The ophthalmic visualization system of claim 13, wherein:
the optical prism is an Abbe-Koenig prism;
the loupe lens assembly includes a lens having a wide field of view; and
the loupe lens assembly is removably coupled to a housing of the image inverter-reverter module.

17. The ophthalmic visualization system of claim 13, wherein:
the image inverter-reverter module is movably coupled to the reduction lens module by a frame; and
the frame includes:
a base attached to the reduction lens module; and
an articulated support arm that is coupled to the image inverter-reverter module.

18. The ophthalmic visualization system of claim 17, wherein:
the articulated support arm is configured to rotate about a first axis that is perpendicular to the optical axis of the ophthalmic microscope; and
the articulated support arm is configured to rotate about a second axis that is perpendicular to the first axis.

19. The ophthalmic visualization system of claim 18, wherein:
the articulated support arm includes a movable cradle that is attached to the image inverter-reverter module;
the movable cradle is configured to translate along a third axis that is perpendicular to the second axis;
the third axis is parallel to the optical axis of the image inverter-reverter module; and
when the image inverter-reverter module is disposed in the deployed position, the third axis is parallel to the optical axis of the ophthalmic microscope.

20. The ophthalmic visualization system of claim 13, wherein:
when the movable reduction lens assembly is disposed in the disengaged position, the optical axis of the movable reduction lens assembly is not aligned with the optical axis of the ophthalmic microscope; and
when the image inverter-reverter module is disposed in the stowed position, the optical axis of the image inverter-reverter module is not aligned with the optical axis of the ophthalmic microscope.
